(19) United States
(12) Patent Application Publication
(10) Pub. No.: US 2020/0282162 A1
(43) Pub. Date: Sep. 10, 2020

(54) SYSTEMS, DEVICES, AND METHODS FOR IMPROVING AMBIENT AIR QUALITY DURING DENTAL, MEDICAL, OR VETERINARY PROCEDURES

(71) Applicant: Pioneer Astronautics, Lakewood, CO (US)

(72) Inventors: Robert Zubrin, Lakewood, CO (US); Stacy L. Carrera, Lakewood, CO (US); Mark Berggren, Lakewood, CO (US)

(21) Appl. No.: 16/811,986

(22) Filed: Mar. 6, 2020

Related U.S. Application Data
(60) Provisional application No. 62/815,975, filed on Mar. 8, 2019.

(51) Int. Cl. A61M 16/00; A61M 16/06; A61M 16/10; A61M 16/08

(52) U.S. Cl. CPC A61M 16/009; A61M 16/06; A61M 16/1075; A61M 16/0891; A61M 2205/3337; A61M 2202/0241; A61M 2202/0283; A61M 2202/062; A61M 2205/366; A61M 2202/0275

(57) ABSTRACT

A novel method and device for the destruction of nitrous oxide in gases such as those resulting from exhaled breath during dental, medical, and veterinary procedures are described. The method employs processing steps including the collection of gases containing constituents such as water vapor, carbon dioxide, oxygen, nitrogen, and nitrous oxide from exhaled breath or from ambient room air, optional removal of moisture from the collected gas, catalytic decomposition of nitrous oxide gas to nitrogen and oxygen, heat exchange to reduce high temperatures in gases exiting the reactor, and sorbents to remove traces of reaction byproducts. Instrumentation and controls are employed to monitor and regulate temperatures, pressures, gas compositions, and flow rates while also providing measures to automatically shut down in the event of off-nominal conditions. The method and device are capable of operating with variable anesthetic or patient exhaled breath flow rates while inducing no significant pressure or vacuum on the patient as they exhale. The method is carried out in a compact device suitable for operation in dental offices, hospitals, and other locations where nitrous oxide is administered as an anesthetic.

### RELATED APPLICATIONS

This application claims the benefit of U.S. Provisional application No. 62/815,975 titled “Systems, Devices, and Methods for Improving Ambient Air Quality During Dental, Medical, or Veterinary Procedures” filed Mar. 8, 2019 which is incorporated herein by reference.

### TECHNICAL FIELD

This disclosure relates to mitigating nitrous oxide released into ambient air during procedures in which nitrous oxide is administered to a patient.

### BACKGROUND

Nitrous oxide is used as an anesthetic during dental, medical, and veterinary procedures, often as a mixture with oxygen in the range of 50:50 by volume. Nitrous oxide used as an anesthetic is not metabolized or consumed during use and is therefore exhaled in amounts equal to those delivered to the patient. As patients exhale, nitrous oxide is directed from a mask to an exhaust hose to direct the exhaled gas away from the patient and medical personnel. Nevertheless, some of the nitrous oxide escapes into the area where it is being administered. The National Institute for Occupational Safety and Health (NIOSH) has established a recommended exposure limit (REL) for nitrous oxide of 25 parts per million as a time-weighted average for the duration of the exposure, and the American Conference of Governmental Industrial Hygienists (ACGIH) has assigned a nitrous oxide threshold limit value (TLV) of 50 ppm for a normal 8-hour work day and 40-hour workweek (Occupational Safety & Health Administration “Occupational Safety and Health Guideline for Nitrous Oxide”). Concentrations of over 100 ppm have been noted in work areas (“Review of Toxicological Data on Nitrous Oxide”, Paul Branptom, Report MGC 153/08/E, European Industrial Gases Association AISBL, Brussels, 2008), which could lead to potential long-term risk to personnel.

Nitrous oxide used in the manner described above is eventually released to the atmosphere. Nitrous oxide is a greenhouse gas, having a lifetime in the atmosphere of greater than 100 years and a global warming potential of about 200 times that of carbon dioxide. New methods and devices for mitigating the amount of nitrous oxide released into the atmosphere are needed.

The present invention is directed toward overcoming one or more of the problems discussed above.

### SUMMARY OF THE INVENTION

Provided herein are methods and devices for the decomposition of anesthetic nitrous oxide in gases such as those resulting from exhaled breath during dental or medical procedures. In some aspects, the methods employ several steps summarized as follows.
Collection of gases:gases may contain constituents such as water vapor, carbon dioxide, oxygen, nitrogen, and nitrous oxide from exhaled breath or from ambient room air. In some aspects, the gases are collected using a fan or blower with appropriate controls to maintain pressure at a fixed level from a patient or other supply and to provide sufficient pressure to feed a nitrous oxide decomposition apparatus.
Optional removal of moisture: moisture can, in some aspects, be removed from the collected gas using a moisture-selective desiccant. In some aspects, the desiccant can be calcium sulfate.
Catalytic decomposition of nitrous oxide gas to nitrogen and oxygen: in some aspects, the catalyst comprises copper, rhodium, ruthenium, or other active metal on an appropriate durable substrate such as alumina, zirconia, or silicate material at temperatures above about 300° C., for example, about 350° C., or about 400° C., or about 450° C.
Heat exchange to reduce the high temperatures of gases exiting the reactor. In some embodiments, heat removed from the gases can be used as an indirect heat exchanger to optionally provide heat for regeneration of the moisture removal desiccant and a radiator for dissipation of remaining process heat to the surroundings.
Removal of trace residual gases: in some aspects, trace residual gases including nitrous oxide and nitrous oxide decomposition byproducts are removed using post-decomposition sorbents.
Optional measurement to detect traces of unreacted nitrous oxide and potential nitrous oxide decomposition byproducts:such byproducts include oxides of nitrogen (NOx) and compounds such as nitric oxide (NO) and nitrogen dioxide (NO₂). In some aspects, the detection is accomplished using in-line sensors.
Venting of treated gases:gases can be vented from the apparatus, for example, to the area in which the apparatus is operated or to a vent directed outdoors.

Instrumentation and controls can be employed to monitor and regulate temperatures, pressures, gas compositions, and flow rates while also providing measures to automatically shut down in the event of off-nominal conditions. The method is carried out in a compact device suitable for operation in dental offices, hospitals, and other locations where nitrous oxide is administered as an anesthetic.

The Nitrous Oxide Decomposition System is designed with pressure controls that ensure patients can breathe normally while collecting exhaled gas (possibly along with some ambient air). The pressure controls are linked to a blower that provides the required suction from exhaled breath to eliminate any back pressure on the patient and also provides the pressure boost required to pass the exhaled breath through the treatment steps described above.

A close-fitting anesthetic mask can facilitate collection of as much of a patient's exhaled breath as possible so that it can be treated in the Nitrous Oxide Decomposition System.

### BRIEF DESCRIPTION OF THE FIGURES

FIG. 1 depicts an exemplary Nitrous Oxide Decomposition System and schematically shows the features described herein for nominal operations.

FIG. 2 depicts the operation of the Nitrous Oxide Decomposition System using simulated patient exhaled breath provided by compressed gas cylinders.

FIG. 3 depicts an example design for a compact nitrous oxide dissociation reactor.

### DESCRIPTION

Before the present systems, devices, and methods are described, it is to be understood that this invention is not limited to particular systems, devices, methods, and experimental conditions described, as such may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, preferred methods and materials are now described.

The decomposition of nitrous oxide to nitrogen and oxygen gases reduces potential human health risks while also mitigating greenhouse gas emissions.

Nitrous oxide is dissociated according to the following exothermic reaction.

N₂O_{(g)}=>N₂+½O₂ΔH=−81.6 kJoules (1)

As shown above, dissociation of N₂O results in the formation of nitrogen and oxygen, the primary components of air. Therefore, dissociation of nitrous oxide to oxygen-rich air (containing one-third by volume of oxygen and two-thirds by volume of nitrogen) eliminates risks to medical personnel while also eliminating release of a potent greenhouse gas to the atmosphere.

Non-catalytic dissociation of nitrous oxide occurs at temperatures in excess of about 565° C. (Matheson Gas Data Book, Fifth Ed, 1971). When pure nitrous oxide gas is dissociated from a starting temperature of 25° C., temperatures of about 1,650° C. can be attained from the strong exothermic dissociation reaction. Therefore, thermal controls are required to hold temperatures in a range suitable for dissociation while protecting hardware from damage.

Catalysts can be used to reduce the temperature at which nitrous oxide decomposes and to minimize the potential formation of byproduct nitrogen oxide gases (NOx) such as nitric oxide (NO) and nitrogen dioxide (NO₂), the formation of which increases at higher temperatures. Catalysts containing metal or metal oxides such as ruthenium, rhodium, nickel, zirconium, copper, and others have been shown to be effective for nitrous oxide dissociation. Dissociation temperatures as low as about 300° C., for example, about 300° C., about 350° C., about 400° C., or about 450° C., can be effective when used with a proper catalyst and hardware configuration. Means for controlling temperature include cooling via passive or forced convection through indirect internal or external cooling gas passages (with heat removal by a radiator). Reactor cooling fins may also be employed to shed heat generated from the dissociation reaction. Alternatively, active cooling via refrigeration, water evaporation, or other means may be employed. In addition, the reactor feed gas can be diluted with room air or reaction product to reduce the temperature resulting from the strong nitrous oxide dissociation exotherm.

Provided herein are methods, systems, and devices for decomposing exhaled nitrous oxide. The methods, systems, and devices are capable of operating with variable anesthetic or patient exhaled breath flow rates while inducing no significant pressure or vacuum on the patient as they exhale. The Nitrous Oxide Decomposition System includes a catalytic reactor system equipped with start-up heaters to achieve the minimum temperature required for dissociation and thermal controls to maintain maximum operating temperatures at target values. Start-up heaters can be shut off once the minimum nitrous oxide dissociation temperature is reached. Despite the strong nitrous oxide dissociation exotherm, some initial gas inlet pre-heating may be required to prevent quenching of the catalyst to temperatures below the minimum requirement.

In some embodiments, a system or device for mitigating exhaled nitrous oxide levels is provided. In some aspects, the system or device comprises: (a) a capture module for capturing exhaled gases; (b) a heater for increasing the temperature of the captured gases; (c) a catalytic nitrous oxide decomposition reactor; and (d) a module for decreasing the temperature of the exhaust gases. In some aspects, the system or device further comprises one or more of the following: (e) a pressure control; (f) a pressure sensor; (g) a desiccant; (h) a heat exchanger; (i) reactor cooling fins; (j) a start up heater; (k) a byproduct gas sensor; and (l) a vent.

In some embodiments, a system or device further comprises one or more of the following: (e) a surge chamber consisting of a tube or other shape closed on one end and open on the other end; (f) a port on the closed end through which tubing from anesthetic exhaust gas is introduced such that the diameter of the port and tubing is sufficient to minimize pressure loss as the anesthetic gas flows into the surge chamber; (g) a port on the open end through which dilution air is introduced such that the diameter of the port is sufficient to minimize pressure loss as the dilution air flows into the surge chamber; (h) a port on the side of the tube or other shape through which tubing is connected to a gas pump such that the diameter of the port and tubing is sufficient to minimize pressure loss as the mixture of anesthetic gas and dilution air flows toward the pump inlet; (i) a manometer or other pressure measurement system attached to the tubing that supplies anesthetic gas to the surge chamber to ensure no significant pressure or vacuum is present in the tubing; (j) a surge chamber of volume sufficient to enable complete collection of anesthetic exhaust gas during temporary periods of high exhaust flow such that no anesthetic gas passes through the dilution air intake to the ambient surroundings; (k) a gas pump of sufficient capacity to draw combined anesthetic gas plus dilution air from the surge chamber and to deliver the gas to the inlet of a nitrous oxide decomposition system at sufficient pressure to overcome system pressure losses; and (l) a gas pump with variable speed control to allow for adjustment of the rate of dilution air flow while still drawing the entire flow of anesthetic exhaust gas.

In some embodiments, a system or device further comprises one or more of the following: (e) reactor fabricated from stainless steel, Inconel, or other alloy suitable for operation in the presence of nitrous oxide, carbon dioxide, oxygen, nitrogen, and water vapor at temperatures up to 700° C.; (f) a reactor of a length:diameter ratio that provides sufficient catalyst volume while providing low pressure drop; (g) a reactor fabricated using microchannel methods to provide a compact configuration with heat exchange channels and catalyst channels; (h) internal heat exchanger configured for counter-current flow consisting of tubing or a series of tubing of sufficient diameter to minimize pressure losses from flowing gas while removing heat from exothermic dissociation of nitrous oxide; (i) internal heat exchanger configured for co-current flow consisting of tubing or a series of tubing of sufficient diameter to minimize pressure losses from flowing gas while removing heat from exothermic dissociation of nitrous oxide; (j) a catalyst containing rhodium, ruthenium, nickel, copper, zirconia, or other elements or compounds active toward nitrous oxide dissociation; (k) a fine screen or other suitable support to retain catalyst particles within the reactor; (l) heaters and controls to pre-heat the reactor and then to maintain the reactor at temperatures to achieve N₂O decomposition; (m) inlet and outlet ports; (n) a heater installed on the connecting line between the internal heat exchanger and the catalyst bed inlet to assist with system preheating and to provide additional temperature control during operation; (o) a radiator installed on the connecting line between the internal heat exchanger and the catalyst bed inlet to assist with heat removal and to provide additional temperature control during operation; (p) a radiator or other cooling device to reduce the temperature of the reactor exhaust gas prior to the next process step; and (q) a sensor to detect concentration of nitrous oxide in the cooled reactor exhaust.

In some aspects, the device or system further comprises a second catalytic reactor after the nitrous oxide destruction reactor to decompose nitrogen oxide compounds. In some aspects, the system additionally comprises one or more of the following: (e) reactor fabricated from stainless steel or other alloy suitable for operation in the presence of nitrous oxide, carbon dioxide, oxygen, nitrogen, and water vapor at temperatures in excess of 100° C.; (f) a catalyst containing rhodium, ruthenium, nickel, copper, zirconia, or other elements or compounds active toward NOx dissociation; (g) heaters and controls to pre-heat the reactor and then to maintain the reactor at temperatures to achieve NOx decomposition; (h) fine screen or other suitable support to retain catalyst particles within the reactor; and (i) inlet and outlet ports.

In some aspects, the device or system further comprises a module to trap nitrogen oxide byproduct gases (NOx) including nitric oxide (NO) and nitrogen dioxide (NO₂) from exhaust gas from a nitrous oxide dissociation reactor. In some aspects, the system further comprises one or more of the following: (e) a sorbent trap fabricated from stainless steel, plastic, or other material suitable for operation in the presence of nitrous oxide, carbon dioxide, oxygen, nitrogen, and water vapor at temperatures below 100° C.; (f) a sorbent containing activated carbon, activated carbon impregnated with sodium hydroxide, potassium hydroxide, or other base suitable for chemically reacting NOx; (g) a sorbent containing sodium or potassium aluminate compounds; (h) a sorbent containing Mayenite; (i) a fine screen or other suitable support to retain sorbent particles within the sorbent trap; (j) inlet and outlet ports; (k) sensors to detect concentrations of NO and NO₂ at the inlet and outlet of the sorbent trap; (l) a second sorbent trap installed to allow for manual or automated switching to the second trap when NO or NO₂ are detected in the first trap outlet; and (m) output from the sensors to alert operators to NO or NO₂ breakthrough and to allow for manual or automated switching to the second trap.

In some aspects, the system or device further comprises one or more of the following: (e) one or more of temperature, flow, pressure, and gas composition sensors; (f) a data acquisition and control system; (g) a user interface to command start up and shut down sequences and to provide system status information, warnings, and alarms; (h) feedback of relevant sensor information to heaters, valves, and flow controllers; (i) a control scheme to automate the procedures for start-up, including valve operations, gas pump operations, and heater operations; (j) a control scheme to automate the procedures for routine operation, including valve operations, gas pump operations, and heater operations; (k) a control scheme to automate the procedures for shut down, including valve operations, gas pump operations, and heater operations; and (l) a control scheme to automate the procedures for off-nominal conditions, including valve operations, gas pump operations, and heater operations.

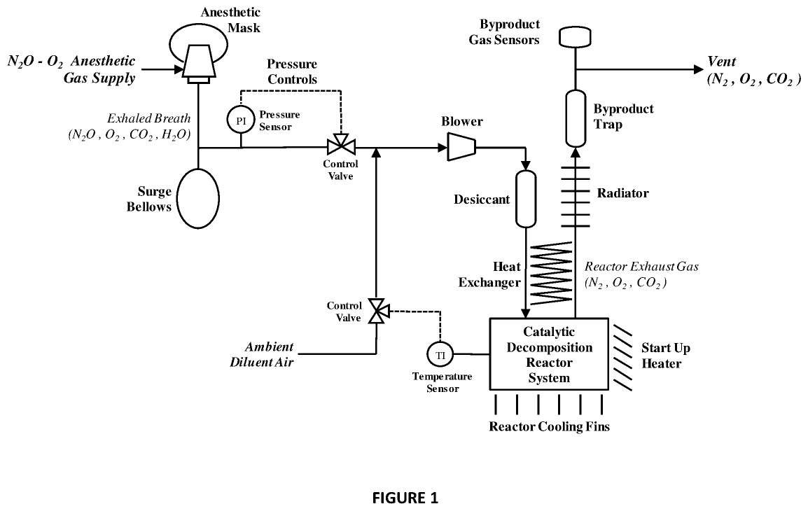

An exemplary Nitrous Oxide Decomposition System is shown in FIG. 1. Prior to operation, the catalytic decomposition reactor system is heated to a minimum temperature required to initiate nitrous oxide decomposition using electric heat. After pre-heating the reactor, exhaled breath containing N₂O, O₂, CO₂, and H₂O is directed to a Nitrous Oxide Decomposition System from the anesthetic mask. A surge bellows or similar device is used to provide a volume to allow for the precise control of the pressure of exhaled breath so that a patient requires no more force for exhalation than would be encountered absent an anesthetic mask. Pressure control is achieved through the regulated flow of exhaled gas through a control valve to the inlet of a blower. The control valve opens or closes in continuous fashion so that the pressure sensor reads a pressure that matches that of the ambient environment. The blower also provides motive force to direct the gases to an optional desiccant (for removal of moisture if desired to prevent potential side reactions and to maintain compatibility with the type of catalyst used downstream) and then to a heat exchanger operating against the hot reactor exhaust gas. The gas delivered to the catalytic decomposition reactor is heated by an indirect heat exchanger only as much as is required to prevent quenching of the reactor system temperature.

The pre-heated gas enters the catalytic decomposition reactor system where nitrous oxide is dissociated into nitrogen and oxygen gas with the attendant release of thermal power. The reactor temperature is controlled using individual controls or a combination of controls consisting of reactor heat dissipation through cooling fins, indirect heat exchange passages, or dilution of the feed gas with ambient air. In one scenario, a reactor temperature sensor is used to control the introduction of dilution air to hold the reactor temperature at a target value. Gas consisting primarily of N₂, O₂, CO₂, and H₂O exiting the reactor system passes first through a heat exchanger to transfer heat to the inlet gas in an amount necessary to pre-heat the inlet gas to prevent quenching of the reactor. Remaining heat is removed from the exhaust gas by radiation to the surroundings. The cooled exhaust gas is then passed through a sorbent bed to remove any traces of nitrogen oxide byproduct gases. A sensor indicates the degree to which byproducts are present. In one scenario, dual sorbent beds are used so that one can be periodically changed while the other is in use. Activated carbon or activated carbon containing a base (such as potassium or sodium hydroxide) along with a small amount of moisture constitutes one example of such a trap. Cooled, clean exhaust gas is then vented from the system to the work area or to another location where it is released to the atmosphere. If a desiccant is used for moisture removal, it is periodically regenerated using waste reaction heat. A two-bed desiccant system is employed to allow for continuous operation of the Nitrous Oxide Decomposition System so that one bed is in operation while the other is being regenerated.

In some embodiments, a system or device is provided for mitigating exhaled nitrous oxide levels. The system or device can comprise a module for capturing exhaled gases, a heat exchanger, a catalytic decomposition reactor, and a byproduct trap.

Provided herein are methods of decomposing exhaled nitrous oxide. In some aspects, the method comprises: (a) capturing exhaled gases; (b) increasing the temperature of the exhaled gases; (c) passing the exhaled gases through a catalytic nitrous oxide decomposition reactor; and (d) decreasing the temperature of the gases exiting the reactor.

In some aspects, the methods further comprise one or more of the following: (e) introducing anesthetic machine exhaust gas into a surge chamber; (f) introducing dilution air from ambient surroundings into the surge chamber; and (g) using a gas pump to draw gases from the surge chamber, thereby allowing anesthetic exhaust gas and dilution air to enter the surge chamber without imposing pressure or vacuum on the patient.

In some aspects, the methods further comprise one or more of the following: (e) passing gases from anesthetic exhaust through a catalyst including rhodium, ruthenium, nickel, copper, zirconia, or other elements or compounds active toward nitrous oxide dissociation; (f) providing a minimum temperature to initiate dissociation of nitrous oxide over a catalyst; (g) providing control to maintain catalyst temperature during nitrous oxide dissociation such that catalyst is not deactivated and excessive concentrations of NOx compounds are prevented; (h) providing a radiator or other means of cooling the reactor exhaust gas prior to the next process step; and (i) providing a sensor to detect nitrous oxide concentration in the exhaust gas.

In some aspects, the methods further comprise operating a second catalytic reactor after the nitrous oxide destruction reactor to decompose nitrogen oxide compounds (NOx) including nitric oxide (NO) and nitrogen dioxide (NO₂) from exhaust gas from a nitrous oxide dissociation reactor to nitrogen and oxygen gas. In some aspects, the methods additionally comprise one or more of the following: (e) passing hot exhaust gas from a nitrous oxide dissociation reactor through a bed of catalyst material consisting of metals such as copper at temperatures in excess of 100° C.; and (f) providing control to maintain the NOx catalytic decomposition temperature at optimum values to achieve maximum NOx decomposition.

In some aspects, the methods further comprise removing nitrogen oxide byproduct gases (NOx) including nitric oxide (NO) and nitrogen dioxide (NO₂) from exhaust gas from a nitrous oxide dissociation reactor. In some aspects, the methods further comprise one or more of the following: (e) passing exhaust gases from a dissociation reactor through a bed of sorbent material consisting of activated carbon or activated carbon impregnated with sodium hydroxide or potassium hydroxide, sodium or potassium aluminate compounds, or Mayenite (Ca₁₂Al₁₄O₃₃) at temperatures below 100° C.; (f) introducing moisture into the sorbent material or in the nitrous oxide dissociation gas to help promote absorption and chemical reaction of NOx compounds at temperatures below 100° C.; and (g) installing two sorbent traps to allow for continued operation when NO or NO₂ is detected in the outlet of one trap by changing the flow to pass through the second trap instead, thereby allowing for the replacement of the first trap.

In some aspects, the methods further comprise one or more of the following: (e) measurement of temperature, flow, pressure, and gas composition; (f) processing instrument measurements from a data acquisition and control system; (g) feedback of relevant sensor information to heaters, valves, and flow controllers; (h) automatically controlling the procedures for start-up, including valve operations, gas pump operations, and heater operations; (i) automatically controlling procedures for routine operation, including valve operations, gas pump operations, and heater operations; (j) automatically controlling the procedures for shut down, including valve operations, gas pump operations, and heater operations; (k) automatically controlling the procedures for off-nominal conditions, including valve operations, gas pump operations, and heater operations; and (l) providing a user interface to command start up and shut down sequences and to provide system status information, warnings, and alarms.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

### EXAMPLES

The following examples are put forth so as to provide those of ordinary skill in the art with a complete disclosure and description of how to make and use the methods and devices described herein, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1

Example 1 is provided to illustrate the Nitrous Oxide Decomposition System operating under nominal conditions, which include the collection and processing of the entire amount of exhaled breath with no pre-dilution. A total flow rate of anesthetic gas of 15 standard liters per minute (SLPM) consisting of a 50:50 mixture (by volume) of N₂O and O₂ is delivered to the patient. Resulting exhaled breath consists of N₂O (at the same average rate of introduction) along with oxygen delivered to the patient minus that amount of oxygen that is converted by metabolism to carbon dioxide (CO₂) and water vapor (H₂O). It is assumed that the exhaled breath is saturated with moisture at a temperature of 37° C., which at sea level results in a concentration of about 6.2 volume percent in the exhaled breath. The following table shows the composition of the exhaled breath before and after nitrous oxide dissociation in the Nitrous Oxide Decomposition System along with the corresponding nominal volumetric and mass flow rates.

TABLE 1
Analysis and Flow Rate of Nitrous Oxide Decomposition System Feed and Exhaust
under Nominal Conditions of Full Treatment with no Gas Dilution.

| | Gas Analysis, Vol % | | Flow Rate, SLPM | | Mass Rate, g/min | |
|---|---|---|---|---|---|---|
| | Inlet | Exhaust | Inlet | Exhaust | Inlet | Exhaust |
| | to N₂O | from N₂O | to N₂O | from N₂O | to N₂O | from N₂O |
| Gas | Dissociation | Dissociation | Dissociation | Dissociation | Dissociation | Dissociation |
| Constituent | Reactor | Reactor | Reactor | Reactor | Reactor | Reactor |
| N₂O | 46.90 | 0.00 | 7.50 | 0.00 | 14.73 | 0.00 |
| N₂ | 0.00 | 37.99 | 0.00 | 7.50 | 0.00 | 9.37 |
| O₂ | 42.21 | 53.19 | 6.75 | 10.50 | 9.64 | 14.99 |
| CO₂ | 4.69 | 3.80 | 0.75 | 0.75 | 1.47 | 1.47 |
| H₂O | 6.19 | 5.02 | 0.99 | 0.99 | 0.80 | 0.80 |
| Total | 100.00 | 100.00 | 15.99 | 19.74 | 26.63 | 26.63 |

The example results shown above do not include the potential presence of by-product gases such as nitrogen oxides, which can be generated in concentrations of nearly zero to 100 ppm or more depending on the reactor system configuration. In practice, byproducts are dissociated using catalytic methods downstream of the reactor system or trapped on sorbents prior to discharge to the work area or atmosphere.

The stated conditions for this example result in an adiabatic reaction temperature of about 1015° C. if the gas fed to the dissociation reactor is pre-heated to about 100° C. to prevent quenching. If gases are preheated to 400° C., the adiabatic temperature is about 1273° C. These temperatures represent the maximum possible reactor temperatures in a fully-insulated system. Heat shed from the reactor during operation leads to substantially lower reaction temperatures. Nevertheless, measures must be taken to avoid operation at temperature beyond materials limitations.

The exothermic nitrous oxide dissociation reaction applied at the rate shown in Table 1 results in a requirement to remove about 460 watts of thermal power. This heat is removed directly from the reactor and shed to the surroundings, is dissipated from hot exhaust gases, or is removed by a combination of thermal management approaches.

### Example 2

Example 2 is provided to illustrate the Nitrous Oxide Decomposition System operating under nominal conditions, which include the collection and processing of the entire amount of exhaled breath but with pre-dilution consisting of introduction of ambient air at a volumetric flow rate equal to the average rate of anesthetic gas administered to the patient. The same total flow rate of anesthetic gas of 15 standard liters per minute (SLPM) consisting of a 50:50 mixture (by volume) of N₂O and O₂ is delivered to the patient as stated for Example 1 above. Resulting exhaled breath consists of N₂O (at the same average rate of introduction) along with oxygen delivered to the patient minus that amount of oxygen that is converted by metabolism to carbon dioxide (CO₂) and water vapor (H₂O). A volumetric flow rate of 15 SLPM of ambient temperature air is mixed with the exhaled breath prior to introduction to the Nitrous Oxide Decomposition System. The following table shows the composition of the exhaled breath containing dilution air before and after nitrous oxide dissociation in the Nitrous Oxide Decomposition System along with the corresponding nominal volumetric and mass flow rates.

TABLE 2
Analysis and Flow Rate of Nitrous Oxide Decomposition System Feed and Exhaust
under Nominal Conditions of Full Treatment with Air Dilution at a Rate Equal to the
Anesthetic Rate.

| | Gas Analysis, Vol % | | Flow Rate, SLPM | | Mass Rate, g/min | |
|---|---|---|---|---|---|---|
| | Inlet | Exhaust | Inlet | Exhaust | Inlet | Exhaust |
| | to N₂O | from N₂O | to N₂O | from N₂O | to N₂O | from N₂O |
| Gas | Dissociation | Dissociation | Dissociation | Dissociation | Dissociation | Dissociation |
| Constituent | Reactor | Reactor | Reactor | Reactor | Reactor | Reactor |
| N₂O | 24.20 | 0.00 | 7.50 | 0.00 | 14.73 | 0.00 |
| N₂ | 38.24 | 55.70 | 11.85 | 19.35 | 14.81 | 24.18 |
| O₂ | 31.95 | 39.29 | 9.90 | 13.65 | 14.13 | 19.49 |
| CO₂ | 2.42 | 2.16 | 0.75 | 0.75 | 1.47 | 1.47 |
| H₂O | 3.19 | 2.85 | 0.99 | 0.99 | 0.80 | 0.80 |
| Total | 100.00 | 100.00 | 30.99 | 34.74 | 45.94 | 45.94 |

The stated conditions for Example 2 result in an adiabatic reaction temperature of about 652° C. if the gas fed to the dissociation reactor is pre-heated to about 100° C. to prevent quenching. If gases are preheated to 400° C., the adiabatic temperature is about 921° C. These temperatures represent the maximum possible reactor temperatures in a fully-insulated system. Heat shed from the reactor during operation leads to substantially lower reaction temperatures. The effect of diluting the gas fed to the Nitrous Oxide Decomposition System with air at a rate equal to the rate anesthetic gas administration is significant, with a reduction of maximum temperature by about 350° C. compared to the case in Example 1.

The exothermic nitrous oxide dissociation reaction applied at the rate shown in Table 2 still results in a requirement to remove about 460 watts of thermal power. In a manner similar to Example 1, this heat is removed directly from the reactor and shed to the surroundings, is dissipated from hot exhaust gases, or is removed by a combination of thermal management approaches.

The Nitrous Oxide Decomposition System can be operated under different scenarios to achieve the same goal of nitrous oxide dissociation with minimal production of byproducts. In one scenario, a compact, high-temperature reactor can be operated to provide rapid nitrous oxide dissociation, but with greater production of NOx byproduct. The gas from this reactor is then passed through a second, lower-temperature reactor where any remaining nitrous oxide is dissociated, and NOx byproducts are catalytically destroyed or optionally, collected on a downstream sorbent. The same category of catalysts that are effective for nitrous oxide dissociation are also often effective for NOx destruction directly or in the presence of a reducing agent.

In another scenario, a single reactor is operated at lower temperatures to achieve nitrous oxide dissociation with minimal formation of NOx byproducts. In this scenario, a reactor of sufficient volume is operated with sufficient retention time to achieve nitrous oxide dissociation with minimal formation of NOx. In all cases, the heat generated from nitrous oxide dissociation must be dissipated in order to control reactor temperatures within target bounds.

A nitrous oxide dissociation reactor may be fabricated from various stainless steels, high-temperature alloys, or nickel. Nickel provides high thermal conductivity to aid removal of heat while also providing some catalytic activity toward nitrous oxide dissociation. Catalysts for the Nitrous Oxide Decomposition System may be in the form of pellets, surface coatings, or other shapes that are compatible with the reactor materials and configuration. Reactor design is tailored to the use of any particular catalyst, each of which has potential limitations related to minimum and maximum operating temperature. For example, rhodium and ruthenium catalysts can provide low-temperature initiation of the nitrous oxide dissociation reaction (in the range of 400° C.). However, at temperatures in excess of 700-800° C., oxides of these metals can become volatile and gradually be evolved from their substrates (depositing in cooler sections of the process downstream of the reactor). Catalysts such as zirconium oxide are capable of operating at very high temperature, but are generally not as active as other catalysts such as rhodium and ruthenium.

### Example 3

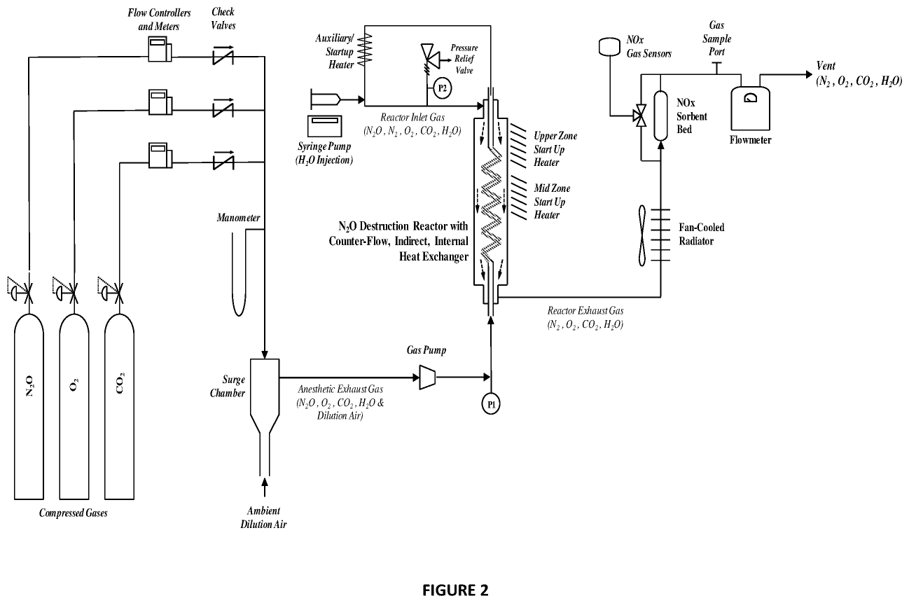

This example illustrates the operation of the Nitrous Oxide Decomposition System using simulated patient exhaled breath provided by compressed gas cylinders. FIG. 2 is a schematic of the system employed for the initial experiments.

Compressed gas cylinders equipped with regulators and flow controllers were used to supply gas in appropriate amounts to represent the nominal exhaust from a nitrous oxide anesthetic machine (see Examples 1 and 2 for flow rates). Check valves were installed to prevent back flow into the gas supply cylinders. The combined dry, simulated anesthetic gas was next fed to a surge chamber. The surge chamber allowed ambient air dilution while at the same time minimizing any vacuum or pressure on the patient. A manometer installed on the simulated anesthetic gas delivery line verified that virtually no suction or pressure was present in the gas line representing the patient breath exhaust. The anesthetic exhaust was drawn into a gas pump along with dilution air from the ambient surroundings. The gas pump can be set to operate at a fixed rate, or it can be adjusted to pull the entire amount of anesthetic gas along with any desired rate of dilution air. The gas pump provided sufficient pressure to convey gases through the Nitrous Oxide Destruction System. The gas pump exhaust was directed to the inlet of a heat exchanger positioned inside the reactor to provide a means of removing reaction heat while pre-heating the anesthetic gas. A relief valve was installed as a safety measure to release excess pressure in the event of off-nominal conditions. Water was injected into the heated gas via a syringe pump operating at a rate to represent moisture present in exhaled breath. The injected water vaporized in the hot gas line. The heated gas exhausting from the internal reactor heat exchanger was then introduced to the reactor catalyst bed. The inlet gas to the catalyst bed was partially cooled so that sufficient heat remained to prevent quenching of the nitrous oxide dissociation reaction. As the anesthetic gas and dilution air passed through the reactor catalyst bed, nitrous oxide was dissociated to oxygen and nitrogen along with small amounts of nitrogen oxide by-product gases. The hot reactor exhaust gas was cooled using a radiator to dissipate reaction heat to the surroundings. Cooled gas was passed through a sorbent to remove nearly all of the nitrogen oxide by-product gases that may have formed. NO and NO₂ sensors to detect nitrogen oxides were installed in a manner that allows for detection of nitrogen oxides at the inlet and exhaust of the NOx sorbent. A gas analysis port located in the reactor exhaust line allowed for collection of samples to determine the concentrations of oxygen, nitrogen, nitrous oxide, and carbon dioxide in the system exhaust. A flow meter was also located in the reactor exhaust line to measure the exhaust gas flow rate. Sensors to measure temperature and pressure were installed at several locations in the Nitrous Oxide Destruction System to facilitate evaluation of performance.

Key elements of the hardware employed in conducting experiments using the schematic shown in FIG. 2 are summarized below. The surge chamber, which is used to capture exhaled nitrous oxide-containing anesthetic gas along with dilution air, was constructed from schedule 40 PVC pipe (3.5-inches outside diameter). Alternatively, a light-weight surge chamber can be fabricated from metal or plastic components. One end of about a 17-inch length of PVC pipe was capped. A fitting through which the simulated anesthetic gas is delivered was installed in the cap. The other end of the surge chamber was left open to the atmosphere to allow inflow of dilution air when the pump was operating. The open end of the surge chamber was fitted with an adapter to reduce the opening to about 1 inch diameter. A length of hose of about 1 inch diameter can be added to the opening to minimize the risk of anesthetic flow exiting the surge chamber through the dilution air inlet without causing significant pressurization or suction on the anesthetic gas feed line that a patient could sense. The dilution air inlet diameter and the length of hose attached to it can be varied to produce the desired effect without imposing pressure or suction on the patient exhaust. The surge chamber employed for the following examples was about 2 liters in volume.

Two ports of about 0.5-inch diameter were installed on the side of the surge chamber for attachment of tubing leading to two gas pumps installed in parallel. The size of the tubing can be adjusted to minimize pressure losses in order to minimize gas pump power and noise levels. For the experiments described herein, two Gast model 22D1180-201-1002 mini diaphragm gas pumps were installed along with speed controllers to allow variation of gas flow rate. Other pumps that are capable of compressing the anesthetic gases plus dilution air to pressures required to generate the desired total flow rate through the Nitrous Oxide Decomposition System would also be suitable. The exhaust from the two pumps was combined in a 0.5-inch diameter tube for delivery to the inlet of the internal heat exchanger of the nitrous oxide dissociation reactor.

The dissociation reactor was fabricated from a 14-inch length of stainless steel tubing (3.5-inches outside diameter; 0.065-inch wall thickness). Alternate alloys of sufficient wall thickness compatible with operation at the target temperatures and pressures are also suitable. An internal heat exchanger consisting of about a 10-foot length of 0.25-inch outside diameter stainless steel tubing was coiled at a diameter of about 1.75-inches diameter with even spacing between coils to allow for filling of catalyst pellets prior to use and for transfer of heat from the catalyst bed to the coiled tubing during operation. Flat end caps were fabricated from stainless steel. Stainless steel compression fittings that allowed for insertion of the indirect heat exchanger coil and for introduction and exhaust of anesthetic gas were welded to each end cap. A catalyst fill/drain port consisting of a 0.75-inch diameter stainless steel compression fitting was installed in the upper portion of the reactor shell. The fill/drain port was fitted with a cap to provide a gas-tight seal during use. Fittings are installed on the reactor shell to accommodate insertion of thermocouples to provide system control and diagnostic data. Additional sample-port fittings were installed along the length of the reactor to facilitate evaluation of reaction extent as a function of various operating parameters. A stainless steel screen was installed on the reactor outlet fitting to retain catalyst pellets.

The reactor was filled with 1.44 kilograms of 0.5-percent rhodium-on-alumina catalyst (Johnson Matthey Type 520). The catalyst was in the form of cylindrical pellets of approximately 0.125-inch diameter and 0.125-inch length.

A heating tape was installed around the circumference of the upper one-third length of the reactor (6-foot length; 0.5-inch width; 468 watts rating at 120 volts). A similar heat tape was installed around the circumference of the middle one-third length of the reactor. Thermocouples were installed under each of the two heat tapes. Variable transformers were used to control the power input to each heat tape. The heat tapes were covered with ceramic fiber insulation. The lower one-third of the reactor was lightly insulated to provide some heat removal during operations.

The connection between the exhaust from the internal reactor heat exchanger and the reactor catalyst bed consisted of a length of 0.25-inch diameter tubing. Fittings for pressure and temperature measurements were installed on the connecting tubing. A pressure relief valve was also installed and set to a pressure of about 25 psi. A 4-inch diameter tightly wound coil consisting of about a 4-foot length of 0.25-inch diameter stainless steel tubing was also installed in the connecting tubing. The coil was wrapped with a heat tape (468 watts) to provide start up and/or pre-heat during operation. The heat tape was controlled using a variable transformer.

A syringe pump (New Era Pump Systems Model NE-300) was installed to inject water at a controlled flow rate just downstream of the heater described above to provide moisture representative of patient breath exhaust.

Dissociation reactor exhaust gas was passed through a radiator consisting of 0.375-inch diameter stainless steel tubing to which copper fins were attached. A fan was installed to provide convection over the radiator surface to enhance heat removal from the exhaust gas.

A NOx sorbent trap was installed on the cooled exhaust gas line along with provisions to take gas samples upstream and downstream of the trap in order to diagnose reactor operations and NOx sorbent performance. The NOx trap consists of an approximate 8-inch length of 1-inch diameter stainless steel tubing. Stainless steel compression fittings were installed on each end with appropriate adapters to connect with inlet and exhaust tubing. A stainless steel screen was installed on the outlet fitting to retain sorbent particles. About 49 grams of potassium hydroxide impregnated activated carbon (American Filtration DAXB-403) were loaded into the trap. The KOH-impregnated activated carbon was in the form of cylindrical pellets of about 0.125-inch diameter and 0.5-inch length. Tubing, valves, and rotameters were installed to direct a small flow of the dissociation reactor exhaust gas from the sorbent trap inlet or outlet to NOx monitors. A NO monitor (GasAlert Extreme model GAXT-N-DL) and NO₂ monitor (Gas Alert Extreme model GAXT-D-DL) were calibrated and used to analyze NOx concentrations at each location.

A dry test meter was installed on the final system exhaust tubing to enable measurement of the gas flow rate to facilitate diagnosis of system performance.

A gas chromatograph (Varian CP-4900 Micro-GC) was used to analyze the concentrations of N₂O, O₂ plus Ar, N₂, and CO₂. This instrument was equipped with four columns to separate and resolve each gas constituent of interest using thermal conductivity detectors. Calibrations were performed and verified for each gas prior to experimentation.

The apparatus as depicted in FIG. 2 and described above was started up, operated, and shut down, producing the nitrous oxide dissociation results described below. The goal of the experiment described in this example was to demonstrate that the methods and device were capable of nearly complete dissociation of nitrous oxide contained in anesthetic machine exhaust containing simulated patient exhaled breath at rates representative of the nominal expected inlet conditions.

The dissociation reactor was first preheated by activating the upper and middle reactor zone heat tapes while flowing air (pulled through the surge chamber) at about 9 standard liters per minute (SLPM) via one of the two gas pumps. After reaching upper reactor zone temperatures of about 300° C. (about one hour after applying start up heat), a gas mixture consisting of about 7.5 SLPM N₂O, 6.75 SLPM O₂, 0.75 SLPM CO₂, and 0.99 SLPM H₂O vapor representing the average expected anesthetic machine exhaust composition was introduced to the surge chamber while both of the gas pumps were set to maximum flow. Heaters were adjusted as needed to provide sufficient gas preheating to achieve nearly complete N₂O dissociation while maintaining maximum reactor temperatures about 600° C. Under these conditions, the dilution air flow was about 14.5 SLPM.

Samples were taken to illustrate the system performance. Results showed that the system exhaust gas flow rate was 34.2 SLPM and contained between 15 and 58 parts per million (ppm) of N₂O (by volume) over the steady operating period of about 33 minutes during which samples were taken. These outlet gas compositions represent N₂O dissociation of 99.97 to 99.99 percent, substantially in excess of a target of 99 percent N₂O destruction (which would be about 0.22 percent N₂O). NO analysis of the inlet and outlet gas from the NOx trap showed 0.0 ppm. NO₂ at the inlet to the trap ranged from about 1.7 to 4 ppm. NO₂ at the outlet of the trap was about 0.7 ppm. Analysis for other gas constituents showed about 55.6 percent N₂, 38.7 percent O₂, and 1.8 percent CO₂.

The maximum system temperature during the steady operation during which sampling took place was about 600° C. (upper reactor shell temperature). During this period, the inlet gas heater was provided with about 49 watts of electric power to boost the inlet gas temperature to about 280° C. (to overcome excessive heat loss through the tubing connecting the exhaust of the internal reactor heat exchanger and the reactor catalyst bed inlet). In addition, the upper reactor heater was operated with about 145 watts of electrical input to prevent quenching in the rhodium catalyst bed.

Additional gas chromatograph samples taken from the upper, middle, and lower nitrous oxide dissociation reactor catalyst zones showed only 42, 28, and 26 ppm N₂O, indicating that the reactor and catalyst bed could be significantly smaller while still achieving nearly complete N₂O conversion.

Pressure drop measured across the internal heat exchanger under conditions specified above was about 5 psi. Pressure drop in the tubing connecting the outlet of the internal reactor heat exchanger and the inlet of the catalyst bed was about 4 psi. Pressure drop across the catalyst bed, NOx trap, and dry test meter was about 5 psi.

Shut down was carried out by maintaining gas pump operation to bring ambient temperature room air through the surge chamber after the flow of simulated anesthetic gas was stopped and by shutting off all heaters.

### Example 4

Additional operations and modifications of the Nitrous Oxide Destruction System apparatus were made to reduce the amount of N₂O dissociation catalyst required to achieve at least 99 percent N₂O, to reduce system pressure drop (to reduce pump power requirement and noise), and to reduce system heat up time. The low ppm N₂O concentrations in the upper zone of the reactor described in Example 3 led to replacing half of the active rhodium catalyst with inert alumina pellets of similar particle size. The active catalyst mass of 719 grams was placed in the upper and middle reactor zones on top of the alumina pellets to allow for the same general pre-heating and start up procedures as described in Example 3. The connecting tubing between the internal reactor heat exchanger and the inlet to the catalyst bed was replaced with a length of 0.5-inch diameter stainless steel tubing to reduce pressure drop compared to the longer length of 0.25-inch diameter tubing plus gas heater used during Example 3. A port was installed in the 0.5-inch diameter tubing for injection of liquid water to simulate moisture in patient breath exhaust upon vaporization. The 0.5-inch diameter connecting tube was wrapped with a heat tape capable of up to 285 watts heat input at 120 volts and insulated to provide additional heat up and/or gas preheating if needed.

The Nitrous Oxide Destruction System was started using the procedures described in Example 3. With an air flow of about 9 SLPM, power was applied to the upper and middle reactor zones as well as to the interconnecting 0.5-inch diameter tubing such that the upper reactor temperature reached 500° C. about 30 minutes after initiation of reactor preheating. Maximum total preheating power for the three heated areas combined was about 950 watts. The flow of simulated anesthetic gas was introduced to the surge chamber at that time. The lower pressure drop resulting from replacement of the 0.25-inch diameter interconnecting tubing with 0.5-inch diameter tubing allowed for the use of only one pump instead of the two pumps employed during Example 3, reducing gas pumping power from about 180 watts to 90 watts.

The flow rates of the simulated anesthetic gas were the same as those cited for Example 3.

Samples taken within 20 minutes after initiating anesthetic gas flow showed 700 ppm N₂O at the system exhaust. Heaters were adjusted as needed to provide sufficient gas preheating to achieve nearly complete N₂O dissociation while avoiding quenching of the catalyst bed and while maintaining maximum reactor temperatures below about 640° C. Under these conditions, the dilution air flow was about 8.6 SLPM. Samples were taken to illustrate the system performance. Results showed that the system exhaust gas flow rate was 27.8 SLPM and contained about 29 parts per million (ppm) of N₂O (by volume) over the sampling period. The outlet gas composition represents N₂O dissociation of 99.99 percent.

Analysis for other gas constituents showed about 53.0 percent N₂, 43.0 percent O₂, and 2.6 percent CO₂. NO analysis of the inlet and outlet gas from the NOx trap showed 7.0 and 6.5 ppm, respectively. NO₂ at the inlet to the trap ranged was about 21 ppm. NO₂ at the outlet of the trap was about 7.0 ppm. The NOx analyses indicated that the KOH-impregnated activated carbon sorbent was allowing breakthrough of the NOx compounds. The NOx trap had operated for an extended duration and had not been changed. Its initial effectiveness demonstrated functionality. However, optimization of the KOH concentration and activated carbon type could lead to much longer life. No attempt was made to perform such optimization during the experiments cited in these examples. Furthermore, other potential sorbents such as sodium or potassium aluminate compounds may also be effective sorbents. Alternatively, direct catalytic dissociation of NOx compounds to nitrogen and oxygen gas may also be suitable.

During the steady operating period when gas samples were acquired, no power was applied to the inlet gas heater. Without heating power applied, the temperature of the preheated simulated anesthetic gas plus dilution air was about 370° C., obtained by the transfer of heat from the reactor contents to the inlet gas via the internal reactor heat exchanger. The upper reactor heater was kept on at a power input of about 26 watts, which was probably not necessary to prevent quenching of the catalytic N₂O dissociation reaction. No other process heat was applied during the sampling period.

Additional gas chromatograph samples taken from the upper, middle, and lower nitrous oxide dissociation reactor catalyst zones showed 255, 184, and 49 ppm N₂O, indicating that the reactor and catalyst bed could again be significantly smaller while still achieving nearly complete N₂O conversion.

Pressure drop measured across the internal heat exchanger and 0.5-inch diameter interconnecting tubing under conditions specified above was about 6 psi. Pressure drop across the catalyst bed, NOx trap, and dry test meter was about 1.5 psi.

Shut down was carried out by maintaining gas pump operation to bring ambient temperature room air through the surge chamber after the flow of simulated anesthetic gas was stopped and by shutting off all heaters.

### Example 5

Additional operation of the Nitrous Oxide Destruction System apparatus was conducted to demonstrate nitrous oxide dissociation under variable rates of anesthetic gas delivery and patient breath exhaust that would be expected in actual practice. The flow rate of nitrous oxide can be expected to vary from 0 to about 28 SLPM, resulting in proportioned total flow of anesthetic gas containing exhaled breath to range from 0 to about 60 SLPM, with an average of about 15 SLPM. To simulate this potential flow variability, the flow controllers were adjusted to deliver gases at rates of 28 SLPM N₂O, 27 SLPM O₂, and 3.0 SLPM CO₂ (four times the average expected rates). A bypass valve was installed on the tubing that delivers the simulated anesthetic gas to the surge chamber, allowing the flow to be periodically diverted to a vent line instead of to the surge chamber. Water flow rate was held at the same average expected rate used in Examples 3 and 4 above while the simulated anesthetic gas was directed to the surge chamber in a cyclic manner. The diverting valve was operated to deliver simulated anesthetic gas to the surge chamber for a nominal 2-second period followed by a nominal 6-second period during which flow was directed to a vent (bypassing the system). This cyclic introduction of simulated anesthetic gas was maintained over a period of 10 minutes while system temperatures, pressures, and gas compositions were monitored. The gas pump speed control was left at the same rate as that for Example 4 above (one pump operating at 100 percent of capacity). This simulated operating profile resulted in brief periods of high-flow-rate anesthetic gas followed by periods of nearly pure air being fed to the Nitrous Oxide Destruction System.

The surge chamber serves the purpose of allowing for periodic high flows of anesthetic gas without discharge of the anesthetic gas through the dilution air inlet. For this example, the gas pump was determined to be operating at a rate of about 31 actual liters per minute (actual volumetric flow rate at the nominal temperature and pressure of gases passing through the surge chamber). On this basis, it was determined that a minimum surge tank volume of about 1.6 liters prevented escape of anesthetic gas through the dilution air inlet port when operating in the manner described above. The surge tank volume was about 2 liters in volume.

One can readily calculate the volume of a surge chamber required to prevent anesthetic gas from discharging through the dilution air inlet port for any given duration of anesthetic gas flowing at a known rate along with knowledge of the gas pump flow rate.

The periodic high flow rate of simulated anesthetic gas can be expected to generate higher temperatures due to the exothermic nature of the N₂O dissociation reaction. However, the relatively short duration of high anesthetic gas flow rate and the subsequent period of nearly pure air flow mitigate this effect. A composite sample of the system exhaust gas taken over a one-minute period during cyclic feed flows showed only 163 ppm N₂O. Reactor temperatures were steady and matched those of non-cyclic anesthetic flow delivered at the average nominal rates prior to cyclic flow experiments. The results showed that the reactor system is capable of handling with flow variabilities of the type described here. Additional control for longer-duration flow variability could also be achieved by adjusting the gas pump speed.

### Example 6

Example 6 is provided as a prophetic example of a further Nitrous Oxide Destruction System embodiment based on the results obtained from experiments described above. The results of gas analyses taken along the length of the reactor and described in Example 4 indicate that further reduction in catalyst mass is possible while still achieving at least 99 percent N₂O dissociation. For example, the concentration of N₂O in the uppermost portion of the catalyst bed during Example 4 was only 255 ppm, which represents about 99.9 percent N₂O dissociation. Approximately 350 grams of catalyst were present in the reactor volume above the upper sample port from which the 255 ppm N₂O analysis was obtained. Therefore, the amount of catalyst could be reduced by at least a factor of two compared to that described for Examples 4 and 5.

Based on thermal analysis and the use of 350 grams of catalyst similar to that used in the examples cited above, a compact reactor design was generated. Note that the compact reactor design described here is based on reactor geometry and catalyst characteristics similar to those described in the examples above. However, alternate reactor designs, such as those for integrated microchannel heat exchangers/catalytic reactors could also be developed to produce an even smaller reactor.

A compact reactor containing an internal heat exchanger and 350 grams of catalyst pellets will be built according to the following design. Note that adjustments to reactor dimensions and heat exchange tubing diameter could be made to optimize such a reactor. The compact reactor described here could be fabricated from a relatively thin wall stainless steel, Inconel, or other suitable alloy with an outside diameter of about 3.5 inches and a catalyst zone of about 3.5 inches length. A thin wall internal heat exchanger can be fabricated from a length of about 28 inches of 0.375-inch outside diameter tubing coiled to fit within the compact reactor to serve the same function as the internal heat exchanger described in the examples above. Sufficient volume remains to fill the reactor with catalyst pellets of a type similar to those described in the examples above.

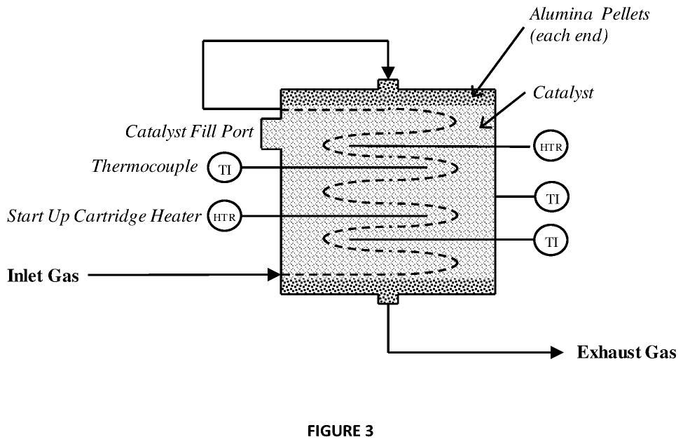

FIG. 3 schematically shows one example of a reactor of the configuration described above. Potential design revisions include variations on the methods and fittings for the internal heat exchanger, catalyst fill port, instrumentation, and heaters. Nevertheless, the basic design takes into consideration the aspects incorporated into the reactor described in Examples 3 through 5.

The body of the reactor consists of a cylindrical, thin-wall, tube or pipe fabricated from stainless steel, Inconel, or other appropriate alloy. In this example, flat end caps are shown. However, the end caps may optionally be fabricated in a dome shape. Fittings are installed to allow for catalyst filling/discharging, thermocouples, heaters, and inlet/outlet gas ports. In the example figure, the reactor internal heat exchange tubing enters from the sides of the reactor, although these could be oriented to pass through the reactor end caps. A layer of alumina pellets are used in the example to aid distribution of gases prior to contact with the active catalyst and to allow for a non-reactive zone in contact with end caps. A screen is installed at ports where the gas is introduced and discharged from the reactor to retain catalyst within the reactor body.

The example compact reactor is designed to achieve nearly complete N₂O dissociation while minimizing pressure drop (and resultant gas pumping power requirement and noise) and while controlling temperatures within the reactor to prevent damage to catalyst or materials of construction.

Thermal analysis showed that air dilution of about 1:1 by volume with the expected average anesthetic gas rate exhaled by a patient would be sufficient along with the use of the internal heat exchanger to raise the temperature of the catalyst bed inlet gas to about 300° C. while holding the maximum reactor temperature to less than 600° C. in a fully-insulated system. In other words, dilution air at about 15 SLPM would be pulled into the surge chamber along with the expected average 15.5 SLPM of exhaled anesthetic gas (7.5 SLPM N₂O, 6.75 SLPM O₂, 0.75 SLPM CO₂, and 0.5 SLPM H₂O vapor).

Although this compact reactor design has lower mass to absorb changes in thermal inputs resulting from potential variable rates of anesthetic gas, the relatively short duration of such variabilities are expected to be compatible with the compact reactor design. In addition, adjustments to the gas pump flow rate can be made to provide greater dilution air flow or lower dilution air flow as appropriate to further control reactor temperatures within selected bounds.

The compact reactor as described is expected to have a mass of approximately 1.3 kilograms including all components described in FIG. 3. About 444 grams of catalyst and alumina pellets would be introduced to the compact reactor.

Based on the mass of the reactor and contents, approximate heat capacities of materials, and the input of about 430 watts heating power, the compact reactor could be brought from a temperature of 25° C. to 400° C. in about 20 minutes. Greater pre-heating power could further reduce heat up rate. In addition, strategic application of heat in the reactor zone where the inlet gas first contacts catalyst could be applied to further reduce pre-heating power input and heat up time by initiating the N₂O dissociation reaction and allowing its heat of reaction to spread through the compact reactor.

The compact reactor and ancillary hardware are capable of being nearly completely automated with respect to start up, operation, and shut down. Appropriate temperature, flow rate, pressure, and gas composition sensors can be incorporated to monitor the system and to trigger alarms based on off-nominal conditions. Automated shut down procedures can be incorporated to stop the Nitrous Oxide Destruction System and vent the anesthetic gas to a safe location away from work area.

What is claimed is:

1. A method of decomposing exhaled nitrous oxide, the method comprising:
(a) capturing exhaled gases;
(b) increasing the temperature of the exhaled gases;
(c) passing the exhaled gases through a catalytic nitrous oxide decomposition reactor; and
(d) decreasing the temperature of the gases exiting the reactor.

2. A system for mitigating exhaled nitrous oxide levels, the system comprising:
(a) a capture module for capturing exhaled gases;
(b) a heater for increasing the temperature of the captured gases;
(c) a catalytic nitrous oxide decomposition reactor; and
(d) a module for decreasing the temperature of the exhaust gases.

3. A device for mitigating exhaled nitrous oxide levels, the device comprising:
(a) a capture device for capturing exhaled gases;
(b) a heater for increasing the temperature of the captured gases;
(c) a catalytic nitrous oxide decomposition reactor; and
(d) a device for decreasing the temperature of the exhaust gases.

4. The system of claim 2, further comprising one or more of the following:
(e) a pressure control;
(f) a pressure sensor;
(g) a desiccant;
(h) a heat exchanger;
(i) reactor cooling fins;
(j) a start up heater;
(k) a byproduct gas sensor; and
(l) a vent.

5. The device of claim 3, further comprising one or more of the following:
(e) a pressure control;
(f) a pressure sensor;
(g) a desiccant;
(h) a heat exchanger;
(i) reactor cooling fins;
(j) a start up heater;
(k) a byproduct gas sensor; and
(l) a vent.

6. A device for mitigating exhaled nitrous oxide levels, comprising a module for capturing exhaled gases, a heat exchanger, a catalytic decomposition reactor, and a byproduct trap.

7. The method of claim 1, further comprising one or more of the following:
(e) introducing anesthetic machine exhaust gas into a surge chamber;
(f) introducing dilution air from ambient surroundings into the surge chamber; and
(g) using a gas pump to draw gases from the surge chamber, thereby allowing anesthetic exhaust gas and dilution air to enter the surge chamber without imposing pressure or vacuum on the patient.

8. The system of claim 2, further comprising one or more of the following:
(e) surge chamber consisting of a tube or other shape closed on one end and open on the other end;
(f) a port on the closed end through which tubing from anesthetic exhaust gas is introduced such that the diameter of the port and tubing is sufficient to minimize pressure loss as the anesthetic gas flows into the surge chamber;
(g) a port on the open end through which dilution air is introduced such that the diameter of the port is sufficient to minimize pressure loss as the dilution air flows into the surge chamber;
(h) a port on the side of the tube or other shape through which tubing is connected to a gas pump such that the diameter of the port and tubing is sufficient to minimize pressure loss as the mixture of anesthetic gas and dilution air flows toward the pump inlet;
(i) a manometer or other pressure measurement system attached to the tubing that supplies anesthetic gas to the surge chamber to ensure no significant pressure or vacuum is present in the tubing;
(j) a surge chamber of volume sufficient to enable complete collection of anesthetic exhaust gas during temporary periods of high exhaust flow such that no anesthetic gas passes through the dilution air intake to the ambient surroundings;
(k) a gas pump of sufficient capacity to draw combined anesthetic gas plus dilution air from the surge chamber and to deliver the gas to the inlet of a nitrous oxide decomposition system at sufficient pressure to overcome system pressure losses; and
(l) a gas pump with variable speed control to allow for adjustment of the rate of dilution air flow while still drawing the entire flow of anesthetic exhaust gas.

9. The device of claim 3, further comprising one or more of the following:
(e) surge chamber module consisting of a tube or other shape closed on one end and open on the other end;
(f) a port on the closed end through which tubing from anesthetic exhaust gas is introduced such that the diameter of the port and tubing is sufficient to minimize pressure loss as the anesthetic gas flows into the surge chamber;
(g) a port on the open end through which dilution air is introduced such that the diameter of the port is sufficient to minimize pressure loss as the dilution air flows into the surge chamber;
(h) a port on the side of the tube or other shape through which tubing is connected to a gas pump such that the diameter of the port and tubing is sufficient to minimize pressure loss as the mixture of anesthetic gas and dilution air flows toward the pump inlet;
(i) a manometer or other pressure measurement device attached to the tubing that supplies anesthetic gas to the surge chamber to ensure no significant pressure or vacuum is present in the tubing;
(j) a surge chamber of volume sufficient to enable complete collection of anesthetic exhaust gas during temporary periods of high exhaust flow such that no anesthetic gas passes through the dilution air intake to the ambient surroundings;
(k) a gas pump of sufficient capacity to draw combined anesthetic gas plus dilution air from the surge chamber and to deliver the gas to the inlet of a nitrous oxide decomposition device at sufficient pressure to overcome system pressure losses; and
(l) a gas pump with variable speed control to allow for adjustment of the rate of dilution air flow while still drawing the entire flow of anesthetic exhaust gas.

10. The method of claim 1, further comprising one or more of the following:
(e) passing gases from anesthetic exhaust through a catalyst including rhodium, ruthenium, nickel, copper, zirconia, or other elements or compounds active toward nitrous oxide dissociation;
(f) providing a minimum temperature to initiate dissociation of nitrous oxide over a catalyst;
(g) providing control to maintain catalyst temperature during nitrous oxide dissociation such that catalyst is not deactivated and excessive concentrations of NOx compounds are prevented;
(h) providing a radiator or other means of cooling the reactor exhaust gas prior to the next process step; and
(i) providing a sensor to detect nitrous oxide concentration in the exhaust gas.

11. The system of claim 2, further comprising one or more of the following:
(e) reactor fabricated from stainless steel, Inconel, or other alloy suitable for operation in the presence of nitrous oxide, carbon dioxide, oxygen, nitrogen, and water vapor at temperatures up to 700° C.;
(f) a reactor of a length:diameter ratio that provides sufficient catalyst volume while providing low pressure drop;
(g) a reactor fabricated using microchannel methods to provide a compact configuration with heat exchange channels and catalyst channels;
(h) internal heat exchanger configured for counter-current flow consisting of tubing or a series of tubing of sufficient diameter to minimize pressure losses from flowing gas while removing heat from exothermic dissociation of nitrous oxide;
(i) internal heat exchanger configured for co-current flow consisting of tubing or a series of tubing of sufficient diameter to minimize pressure losses from flowing gas while removing heat from exothermic dissociation of nitrous oxide;
(j) a catalyst containing rhodium, ruthenium, nickel, copper, zirconia, or other elements or compounds active toward nitrous oxide dissociation;
(k) a fine screen or other suitable support to retain catalyst particles within the reactor;
(l) heaters and controls to pre-heat the reactor and then to maintain the reactor at temperatures to achieve N₂O decomposition;
(m) inlet and outlet ports;
(n) a heater installed on the connecting line between the internal heat exchanger and the catalyst bed inlet to assist with system preheating and to provide additional temperature control during operation;
(o) a radiator installed on the connecting line between the internal heat exchanger and the catalyst bed inlet to assist with heat removal and to provide additional temperature control during operation;
(p) a radiator or other cooling device to reduce the temperature of the reactor exhaust gas prior to the next process step; and
(q) a sensor to detect concentration of nitrous oxide in the cooled reactor exhaust.

12. The device of claim 3, further comprising one or more of the following:
(e) a reactor fabricated from stainless steel, Inconel, or other alloy suitable for operation in the presence of nitrous oxide, carbon dioxide, oxygen, nitrogen, and water vapor at temperatures up to 700° C.;
(f) a reactor of a length:diameter ratio that provides sufficient catalyst volume while providing low pressure drop;
(g) a reactor fabricated using microchannel methods to provide a compact configuration with heat exchange channels and catalyst channels;
(h) internal heat exchanger configured for counter-current flow consisting of tubing or a series of tubing of sufficient diameter to minimize pressure losses from flowing gas while removing heat from exothermic dissociation of nitrous oxide;
(i) internal heat exchanger configured for co-current flow consisting of tubing or a series of tubing of sufficient diameter to minimize pressure losses from flowing gas while removing heat from exothermic dissociation of nitrous oxide;
(j) catalyst containing rhodium, ruthenium, nickel, copper, zirconia, or other elements or compounds active toward nitrous oxide dissociation;
(k) a fine screen or other suitable support to retain catalyst particles within the reactor;
(l) one or more heaters and/or controls to pre-heat the reactor and then to maintain the reactor at temperatures to achieve N₂O decomposition;
(m) inlet and outlet ports;
(n) a heater installed on the connecting line between the internal heat exchanger and the catalyst bed inlet to assist with system preheating and to provide additional temperature control during operation
(o) a radiator installed on the connecting line between the internal heat exchanger and the catalyst bed inlet to assist with heat removal and to provide additional temperature control during operation;
(p) a radiator or other cooling device to reduce the temperature of the reactor exhaust gas prior to the next process step; and
(q) a sensor to detect concentration of nitrous oxide in the cooled reactor exhaust.

13. The method of claim 1, further comprising operating a second catalytic reactor after the nitrous oxide destruction reactor to decompose nitrogen oxide compounds (NOx) including nitric oxide (NO) and nitrogen dioxide (NO₂) from exhaust gas from a nitrous oxide dissociation reactor to nitrogen and oxygen gas, the method additionally comprising one or more of the following:
(e) passing hot exhaust gas from a nitrous oxide dissociation reactor through a bed of catalyst material consisting of metals such as copper at temperatures in excess of 100° C.; and
(f) providing control to maintain the NOx catalytic decomposition temperature at optimum values to achieve maximum NOx decomposition.

14. The system of claim 2, further comprising a second catalytic reactor after the nitrous oxide destruction reactor to decompose nitrogen oxide compounds, the system additionally comprising one or more of the following:
(e) reactor fabricated from stainless steel or other alloy suitable for operation in the presence of nitrous oxide, carbon dioxide, oxygen, nitrogen, and water vapor at temperatures in excess of 100° C.;
(f) catalyst containing rhodium, ruthenium, nickel, copper, zirconia, or other elements or compounds active toward NOx dissociation;
(g) heaters and controls to pre-heat the reactor and then to maintain the reactor at temperatures to achieve NOx decomposition;
(h) fine screen or other suitable support to retain catalyst particles within the reactor; and
(i) inlet and outlet ports.

15. The device of claim 3, further comprising a second catalytic reactor after the nitrous oxide destruction reactor to decompose nitrogen oxide compounds, the device comprising one or more of the following:
(e) a reactor fabricated from stainless steel or other alloy suitable for operation in the presence of nitrous oxide, carbon dioxide, oxygen, nitrogen, and water vapor at temperatures in excess of 100° C.;
(f) a catalyst containing rhodium, ruthenium, nickel, copper, zirconia, or other elements or compounds active toward NOx dissociation;
(g) heaters and controls to pre-heat the reactor and then to maintain the reactor at temperatures to achieve NOx decomposition;
(h) a fine screen or other suitable support to retain catalyst particles within the reactor; and
(i) inlet and outlet ports.

16. The method of claim 1, further comprising removing nitrogen oxide byproduct gases (NOx) including nitric oxide (NO) and nitrogen dioxide (NO₂) from exhaust gas from a nitrous oxide dissociation reactor, the method further comprising one or more of the following:
(e) passing exhaust gases from a dissociation reactor through a bed of sorbent material consisting of activated carbon or activated carbon impregnated with sodium hydroxide or potassium hydroxide, sodium or potassium aluminate compounds, or Mayenite (Ca₁₂Al₁₄O₃₃) at temperatures below 100° C.;
(f) introducing moisture into the sorbent material or in the nitrous oxide dissociation gas to help promote absorption and chemical reaction of NOx compounds at temperatures below 100° C.; and
(g) installing two sorbent traps to allow for continued operation when NO or NO₂ is detected in the outlet of one trap by changing the flow to pass through the second trap instead, thereby allowing for the replacement of the first trap.

17. The system of claim 2, further comprising a module to trap nitrogen oxide byproduct gases (NOx) including nitric oxide (NO) and nitrogen dioxide (NO₂) from exhaust gas from a nitrous oxide dissociation reactor, the system further comprising one or more of the following:
(e) a sorbent trap fabricated from stainless steel, plastic, or other material suitable for operation in the presence of nitrous oxide, carbon dioxide, oxygen, nitrogen, and water vapor at temperatures below 100° C.;
(f) a sorbent containing activated carbon, activated carbon impregnated with sodium hydroxide, potassium hydroxide, or other base suitable for chemically reacting NOx;
(g) a sorbent containing sodium or potassium aluminate compounds;
(h) a sorbent containing Mayenite;
(i) a fine screen or other suitable support to retain sorbent particles within the sorbent trap;
(j) inlet and outlet ports;
(k) sensors to detect concentrations of NO and NO₂ at the inlet and outlet of the sorbent trap;
(l) a second sorbent trap installed to allow for manual or automated switching to the second trap when NO or NO₂ are detected in the first trap outlet; and
(m) output from the sensors to alert operators to NO or NO₂ breakthrough and to allow for manual or automated switching to the second trap.

18. The device of claim 3, further comprising a module to trap nitrogen oxide byproduct gases (NOx) including nitric oxide (NO) and nitrogen dioxide (NO₂) from exhaust gas from a nitrous oxide dissociation reactor, the device further comprising one or more of the following:
(e) a sorbent trap fabricated from stainless steel, plastic, or other material suitable for operation in the presence of nitrous oxide, carbon dioxide, oxygen, nitrogen, and water vapor at temperatures below 100° C.;
(f) a sorbent containing activated carbon, activated carbon impregnated with sodium hydroxide, potassium hydroxide, or other base suitable for chemically reacting NOx;
(g) a sorbent containing sodium or potassium aluminate compounds;
(h) a sorbent containing Mayenite;
(i) a fine screen or other suitable support to retain sorbent particles within the sorbent trap;
(j) inlet and outlet ports;
(k) sensors to detect concentrations of NO and NO₂ at the inlet and outlet of the sorbent trap;
(l) a second sorbent trap installed to allow for manual or automated switching to the second trap when NO or NO₂ are detected in the first trap outlet; and
(m) output from the sensors to alert operators to NO or NO₂ breakthrough and to allow for manual or automated switching to the second trap.

19. The method of claim 1, further comprising one or more of the following:
(e) measurement of temperature, flow, pressure, and gas composition;
(f) processing instrument measurements from a data acquisition and control system;
(g) feedback of relevant sensor information to heaters, valves, and flow controllers;
(h) automatically controlling the procedures for start-up, including valve operations, gas pump operations, and heater operations;
(i) automatically controlling procedures for routine operation, including valve operations, gas pump operations, and heater operations;
(j) automatically controlling the procedures for shut down, including valve operations, gas pump operations, and heater operations;
(k) automatically controlling the procedures for off-nominal conditions, including valve operations, gas pump operations, and heater operations; and
(l) providing a user interface to command start up and shut down sequences and to provide system status information, warnings, and alarms.

20. The system of claim 2, further comprising one or more of the following:
(e) one or more of temperature, flow, pressure, and gas composition sensors;
(f) a data acquisition and control system;
(g) a user interface to command start up and shut down sequences and to provide system status information, warnings, and alarms;
(h) feedback of relevant sensor information to heaters, valves, and flow controllers;
(i) a control scheme to automate the procedures for start-up, including valve operations, gas pump operations, and heater operations;
(j) a control scheme to automate the procedures for routine operation, including valve operations, gas pump operations, and heater operations;
(k) a control scheme to automate the procedures for shut down, including valve operations, gas pump operations, and heater operations; and
(l) a control scheme to automate the procedures for off-nominal conditions, including valve operations, gas pump operations, and heater operations.

21. The device of claim 3, further comprising a module to automatically control a nitrous oxide destruction system, the device further comprising:
(e) one or more of temperature, flow, pressure, and gas composition sensors;
(f) a data acquisition and control system;
(g) a user interface to command start up and shut down sequences and to provide system status information, warnings, and alarms; feedback of relevant sensor information to heaters, valves, and flow controllers;
(h) a control scheme to automate the procedures for start-up, including valve operations, gas pump operations, and heater operations;
(i) a control scheme to automate the procedures for routine operation, including valve operations, gas pump operations, and heater operations;
(j) a control scheme to automate the procedures for shut down, including valve operations, gas pump operations, and heater operations; and
(k) a control scheme to automate the procedures for off-nominal conditions, including valve operations, gas pump operations, and heater operations.
